(19) United States
(12) Patent Application Publication
(10) Pub. No.: US 2026/0090723 A1
(43) Pub. Date: Apr. 2, 2026

(54) DISPOSABLE FIBER-OPTIC SENSOR DEVICE FOR ASSESSING TISSUE BIOMECHANICAL PROPERTIES

(71) Applicant: Washington University, St. Louis, MO (US)

(72) Inventors: Song Hu, St. Louis, MO (US); Jingwei Ling, St. Louis, MO (US); Shuo Yang, St. Louis, MO (US)

(73) Assignee: Washington University, St. Louis, MO (US)

(21) Appl. No.: 19/347,485

(22) Filed: Oct. 1, 2025

Related U.S. Application Data
(60) Provisional application No. 63/701,830, filed on Oct. 1, 2024.

(51) Int. Cl. A61B 5/00; G01J 3/26

(52) U.S. Cl. CPC A61B 5/0084; A61B 5/0051; A61B 5/0075; G01J 3/26; A61B 2560/063; A61B 2562/0233; A61B 2562/164

(57) ABSTRACT

Among the various aspects of the present disclosure is the provision of a disposable fiber optic sensor device for assessing tissue biomechanical properties. Disclosed herein is a fiber-optic sensor device that includes an optical fiber with a fiber tip, a displacement driver, and an interrogation system. A method to measure a biomechanical property of a tissue is also disclosed, which includes using a fiber optic sensor device to compress the targeted tissue with predefined displacement and frequency, where the counterforce deforms the soft cavity and, in turn, modulates the interference pattern is also disclosed.

### CROSS-REFERENCE TO RELATED APPLICATIONS

This application claims priority from U.S. Provisional Application Ser. No. 63/701,830 filed on Oct. 1, 2024, which is incorporated herein by reference in its entirety.

### STATEMENT REGARDING FEDERALLY SPONSORED RESEARCH OR DEVELOPMENT

This invention was made with government support under AT012283 awarded by the National Institutes of Health. The government has certain rights in the invention.

### MATERIAL INCORPORATED-BY-REFERENCE

Not applicable.

### FIELD OF THE INVENTION

The present disclosure generally relates to a fiber-optic sensor for assessing tissue biomedical properties.

### BACKGROUND OF THE INVENTION

Viscoelasticity is a crucial biomechanical parameter for the clinical diagnosis of important diseases, including breast cancer and chronic myofascial pain. A common clinical practice is palpation, which is subjective and varies among physicians. To quantitively evaluate the elasticity of soft tissues, elastography was invented. Existing elastography technologies, including magnetic resonance elastography (MRE), ultrasound elastography (UE), and optical coherent elastography (OCE), are widely used. However, each method has its limitations. Although MRE and UE can image tissue elasticity up to tens of centimeters deep, the spatial resolution is limited to millimeters. OCE has a microscopic resolution, but its penetration is limited to millimeters.

### SUMMARY OF THE INVENTION

Among the various aspects of the present disclosure is the provision of a disposable fiber optic sensor device for assessing tissue biomechanical properties.

Briefly, therefore, the present disclosure is directed to a fiber-optic biomechanical sensor, fiber-optic biomechanical sensing systems that include the fiber-optic biomechanical sensor, and methods of use thereof.

The present teachings include a fiber-optic biomechanical sensing system. The system includes a biomechanical sensor, a light source optically coupled to the biomechanical sensor, a vibrational indentation module mechanically coupled to the biomechanical sensor, and a spectrometer optically coupled to the fiber optic of the biomechanical sensor. The biomechanical sensor includes a fiber optic with opposed proximal and distal ends, and a deformable Fabry-Perot interferometer optically coupled to the distal end of the optic fiber. The light source is optically coupled to the biomechanical sensor at the proximal end of the optic fiber. The vibrational indentation module is mechanically coupled to the biomechanical sensor, and is configured to periodically advance and retract the biomechanical sensor along a proximal-distal axis at a predetermined frequency and displacement. The spectrometer is optically coupled to the fiber optic of the biomechanical sensor, and is configured to receive a plurality of optical signals from the deformable Fabry-Perot interferometer. The optical signals encode interference patterns generated by the deformable Fabry-Perot interferometer in response to the periodic displacements of the biomechanical sensor by the vibrational indentation module. In some aspects, the optical fiber is a polymer-coated glass optical fiber. In some aspects, the Fabry-Perot interferometer includes a deformable cavity comprising a proximal surface optically coupled to the distal end of the fiber optic and a distal surface opposite the proximal surface, a first reflective coating covering at least a portion of the proximal surface, the first reflective coating configured to reflect a first portion of light from the light source at the distal end of the optic fiber and further configured to transmit a second portion of light from the light source to the distal surface, and a second reflective coating covering at least a portion of the distal surface, the second reflective coating configured to reflect the second portion of light back through the first reflective coating and optic fiber, wherein the reflected first and second portions of light form an interference pattern propagating through the optic fiber to the spectrometer. In some aspects, the deformable cavity comprises a soft and transparent material selected from polydimethylsiloxane (PDMS), a hydrogel, and an optical adhesive. In some aspects, the first and second reflective coatings comprise at least one dielectric material. In some aspects, the dielectric materials of the first and second reflective coatings are selected independently from titanium dioxide (TiO₂), zinc oxide (ZnO), and any combination thereof. In some aspects, the first and second reflective coatings comprise thin dielectric coatings formed using a sol-gel method to increase the light reflectivity. In some aspects, the light source comprises a broadband light source selected from a light-emitting diode (LED), a superluminescent diode (SLED), and a lamp. In some aspects, the interference patterns generated by the deformable Fabry-Perot interferometer are modulated by changes in a cavity thickness between the proximal and distal surfaces of the deformable cavity induced by compressions associated with the periodic displacements of the biomechanical sensor by the vibrational indentation module. In some aspects, the biomechanical sensor is disposable and autoclavable for sterilization to facilitate clinical translation. In some aspects, the biomechanical sensor is mass-producible. In some aspects, the vibrational indentation module fiber optic device comprises a piezo actuator operatively coupled to a controller, the controller comprising a voltage modulator. In some aspects, the voltage modulator is configured to produce a modulated voltage at a predetermined frequency or shape to actuate the periodic displacements of the piezo actuator. In some aspects, the spectrometer is a high-speed spectrometer. In some aspects, the biomechanical sensor is further configured to be deployed through a standard 25-gauge injection needle. In some aspects, the biomechanical sensor further comprises a cross-sectional diameter of about 140 μm.

In another aspect, a method to measure at least one biomechanical property of a tissue is disclosed. The methods include the system as described above, positioning the deformable Fabry-Perot interferometer of the biomechanical sensor against the tissue, periodically advancing and retracting the biomechanical sensor along a proximal-distal axis at a predetermined frequency and displacement while delivering light from the light source to generate a series of interference patterns, detecting the series of interference patterns at the spectrometer, and transforming the series of interference patterns into the at least one biomechanical property of the tissue. In some aspects, the at least one biomechanical property is selected from stiffness, viscosity, and any combination thereof. In some aspects, the at least one biomechanical property is recorded in real-time. In some aspects, positioning the deformable Fabry-Perot interferometer of the biomechanical sensor against the tissue comprises deploying the biomechanical sensor through a standard injection needle.

Other objects and features will be in part apparent and in part pointed out hereinafter.

### DESCRIPTION OF THE DRAWINGS

Those of skill in the art will understand that the drawings, described below, are for illustrative purposes only. The drawings are not intended to limit the scope of the present teachings in any way.

FIG. 1 is a schematic diagram of the fiber-optic sensor and corresponding interrogation system. SLD: a super luminescent diode. IMG: Index matching gel, which is used to eliminate reflection at the fiber end.

FIG. 2 is a schematic diagram of the vibrational indentation module with sensor-connecting apparatus. The Indentation generator is connected to a linear stage, which guides the probe toward the measuring area. The fiber-optic sensor is installed on the Indentation generator through a quick-release fiber connector.

FIG. 3A is a pair of graphs of deformation amplitude of the soft cavity, which show larger amplitude in the stretched muscle (bottom graph) compared to the relaxed muscle (top graph).

FIG. 3B is a schematic diagram of the structure of the stiffness sensor. Between the flat fiber tip and the soft cavity and between the soft cavity and the light-absorbing layer there are reflective coatings.

FIG. 3C is a microscopic photo of a stiffness probe.

FIG. 4A is a schematic diagram of the probe of the present disclosure in contact with muscle for use in fiber-optic micro-palpation.

FIG. 4B is a flow diagram of the fiber-optic micro-palpation methods, which starts with a pre-defined vibration of known frequency and amplitude, continues with indentation on the tissue and readout from the force sensor to provide mechanical properties such as stiffness.

FIG. 5 is a schematic showing the functionality of a fiber-tip Fabry-Perot (FP) force sensor.

FIG. 6 is a schematic of the method of fabricating an FP force sensor.

FIG. 7A is a schematic of the FP force sensor interrogation system of the present disclosure that includes a SLED, spectrometer, 2×1 SM fiber coupler, and a fiber sensor.

FIG. 7B is a pair of graphs showing an example of a measured spectrum and its Fourier transform.

FIG. 8A is a schematic of the testing setup for the PDMS phantom test.

FIG. 8B is a graph of the sensor response under palpation (7 μm, 6 Hz).

FIG. 8C is a graph showing how OPD changes at different palpation amplitudes.

FIG. 9A is a photo of the experimental setup for the in vivo validation of the force sensor in rats.

FIG. 9B is a graph comparing the OPD change over time for relaxed muscles and stretched muscles under palpation at 10 Hz.

FIG. 9C is a graph comparing OPD change in relaxed and stretched muscles under palpation at 10 Hz.

FIG. 10A is a photo of the packaged sterilized probe designed for single use.

FIG. 10B is a graph showing the interference pattern of the probe before and after autoclave.

FIG. 11A is a schematic showing the concept of proximity sensing from the sensor probe.

FIG. 11B is a time series of the A-lines in a rat hindlimb, showing real-time feedback with micrometer level accuracy.

FIG. 12A is a photo of the clinical deployment of the probe of the fiber sensor probe of the present disclosure.

FIG. 12B is a graph of the OPD time-domain signal under a 2 μm 2 Hz sinusoidal palpation for 5 seconds.

FIG. 12C is a graph of the Fourier transform of the time-domain signal seen in FIG. 12B.

FIG. 13A is a pair of photos of an improved fiber stiffness sensing probe with reduced size (top) and improved strength (bottom). Scale bar: 100 μm.

FIG. 13B is a graph of sensor readout with a 250 Hz sinusoidal stimulation where the inset plots the corresponding Fourier transform spectrum.

FIG. 13C is a real-time distance measurement by optical coherent detection with the improved sensing fiber seen in FIG. 13A.

FIG. 14A is a schematic of the multi-functional fiber probe design.

FIG. 14B is a detailed schematic of the fiber probe head. PAM: photoacoustic; SHG: second-harmonic generation; FPI: Fabry-Perot interferometer; DM: dielectric mirror; US: ultrasonic.

FIG. 15 is a schematic of the optical system associated with the multi-functional fiber probe. DM: dichroic mirror; M: mirror; L: lens; F: filter; VOA: variable optical attenuator; HP: half-wave plate; (P)BS: (polarization) beam splitter; Obj: objective lens.

### DETAILED DESCRIPTION OF THE INVENTION

The present disclosure is based, at least in part, on the discovery that a fiber-optic biomechanical sensor can be fabricated that provides highly localized assessments of biomechanical properties in deep tissue at high spatial resolution. As shown herein, a disposable through-needle fiber-optic sensor for in-vivo assessment of deep-tissue biomechanical properties is described.

In some aspects, the present disclosure provides an optical fiber, a displacement driver, and an interrogation system. The fiber-optic device can consist of a polymer-coated glass optical fiber and a Fabry-Perot interferometer on the fiber tip. The cavity of the Fabry-Perot interferometer can be formed by soft and transparent materials, including Polydimethylsiloxane (PDMS), Hydrogel, or optical adhesives. Thin dielectric coatings, including titanium dioxide (TiO₂) or zinc oxide (ZnO), can be fabricated on the two sides of the cavity via the sol-gel method to increase the light reflectivity. An amount of broadband light can be launched through the fiber, reach the Fabry-Perot interferometer, and can be partially reflected at the two thin dielectric layers. The two reflected beams can form an interference pattern where the periodicity depends on the cavity thickness. During the measurement, the fiber probe can compress the targeted tissue with predefined displacement and frequency, where the counterforce deforms the soft cavity and, in turn, modulates the interference pattern. Key biomechanical properties of the tissue, including stiffness and viscosity, can be derived from the interference pattern recorded in real-time. The sensor can be seamlessly deployed through standard injection needles. The sensor can be disposable and autoclavable for sterilization to facilitate clinical translation and can be mass-produced.

The fiber device can be driven by an apparatus comprised of a piezo with a controller connected to an external voltage modulator. The modulated voltage of a specific frequency or shape can actuate the displacement generator. Key components of the interrogation system can include a broadband light source, such as a light-emitting diode (LED), a superluminescent diode (SLED) or a lamp, and a high-speed spectrometer.

### KITS

Also provided are kits. Such kits can include a fiber-optic biomechanical sensor and/or associated supplies as described herein and, in certain embodiments, instructions for administration. Such kits can facilitate the performance of the methods described herein. When supplied as a kit, the different components of the composition can be packaged in separate containers and assembled or admixed immediately before use. Components include, but are not limited to an optical fiber sensor and other components described herein. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing activity of the components.

For example, sealed glass ampules may contain fiber optic components packaged under a neutral non-reacting gas, such as nitrogen. Ampules may consist of any suitable material, such as glass, organic polymers, such as polycarbonate, polystyrene, ceramic, metal or any other material typically employed to hold sterile equipment. Other examples of suitable containers include bottles that may be fabricated from similar substances as ampules, and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or other substrate, and/or may be supplied as an electronic-readable medium or video. Detailed instructions may not be physically associated with the kit; instead, a user may be directed to an Internet website specified by the manufacturer or distributor of the kit.

A control sample or a reference sample as described herein can be a sample from a healthy subject. A reference value can be used in place of a control or reference sample, which was previously obtained from a healthy subject or a group of healthy subjects. A control sample or a reference sample can also be a sample with a known amount of a detectable compound or a spiked sample.

The methods and algorithms of the invention may be enclosed in a controller or processor. Furthermore, methods and algorithms of the present invention, can be embodied as a computer-implemented method or methods for performing such computer-implemented method or methods, and can also be embodied in the form of a tangible or non-transitory computer-readable storage medium containing a computer program or other machine-readable instructions (herein “computer program”), wherein when the computer program is loaded into a computer or other processor (herein “computer”) and/or is executed by the computer, the computer becomes an apparatus for practicing the method or methods. Storage media for containing such computer programs include, for example, floppy disks and diskettes, compact disk (CD)-ROMs (whether or not writeable), DVD digital disks, RAM and ROM memories, computer hard drives and back-up drives, external hard drives, “thumb” drives, and any other storage medium readable by a computer. The method or methods can also be embodied in the form of a computer program, for example, whether stored in a storage medium or transmitted over a transmission medium such as electrical conductors, fiber optics or other light conductors, or by electromagnetic radiation, wherein when the computer program is loaded into a computer and/or is executed by the computer, the computer becomes an apparatus for practicing the method or methods. The method or methods may be implemented on a general-purpose microprocessor or on a digital processor specifically configured to practice the process or processes. When a general-purpose microprocessor is employed, the computer program code configures the circuitry of the microprocessor to create specific logic circuit arrangements. Storage medium readable by a computer includes medium being readable by a computer per se or by another machine that reads the computer instructions for providing those instructions to a computer for controlling its operation. Such machines may include, for example, machines for reading the storage media mentioned above.

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing from the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1—A Disposable Through-Needle Fiber-Optic Sensor for In-Vivo Assessment of Deep-Tissue Biomechanical Properties

More than 85% of the general public is affected by myofascial pain sometime in their lives. However, current diagnostic approaches are subjective, leading to both undertreatment and overtreatment, and continued suffering. Trigger points, which are contracted or overstretched muscle fibers centimeters under the skin surface, play a role in myofascial pain, and their analysis is key to providing diagnostic information. Existing approaches include ultrasound elastography and magnetic resonance elastography, however, these approaches only provide macroscopic information. Therefore, providing microscopic biomechanical insights into trigger points is a potential complementary biomarker for myofascial pain that presents a challenge to assess.

Tissue biomechanical properties serve as crucial diagnostic biomarkers. While various elastography techniques have been developed to assess those properties in deep tissue, the resolution is limited to millimeters, which hinders detailed disease examination.

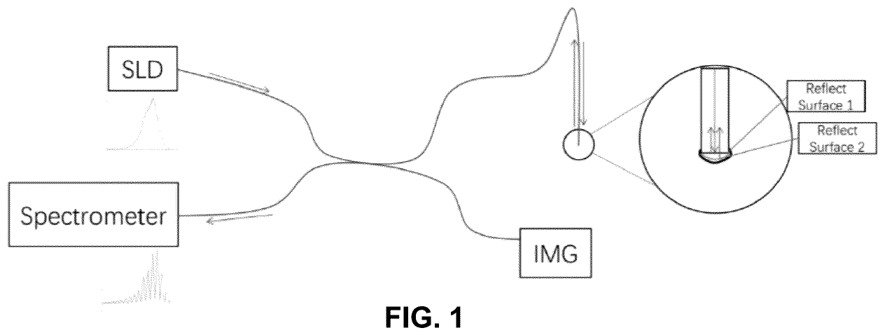

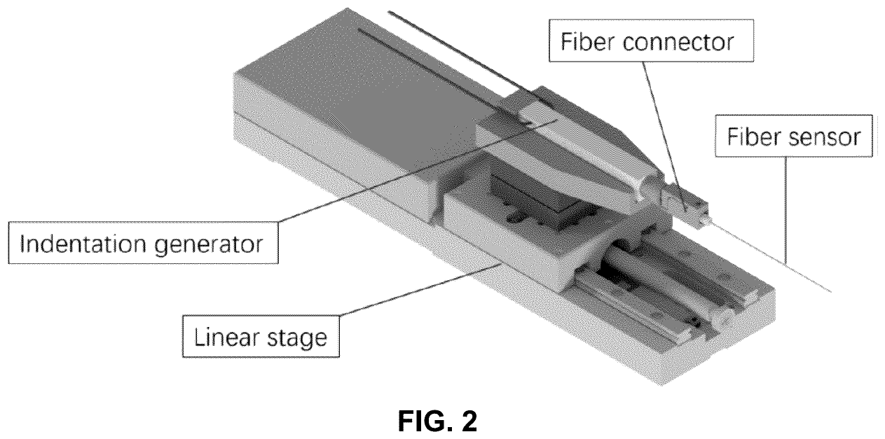

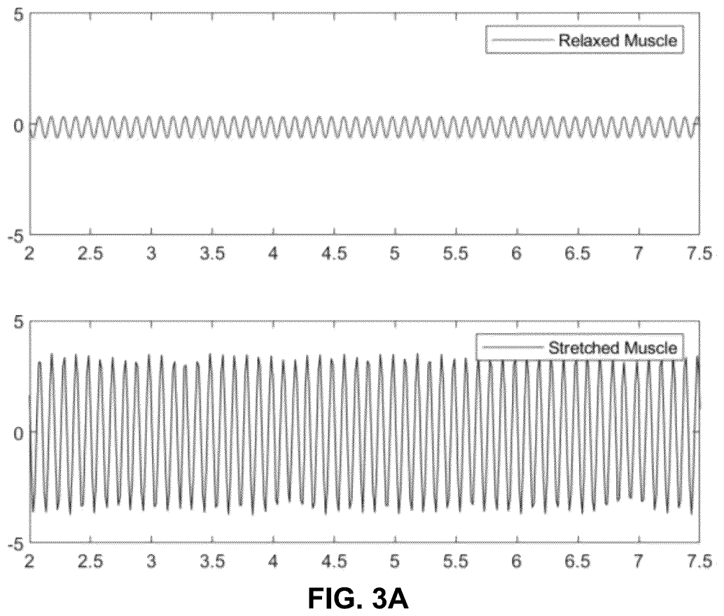

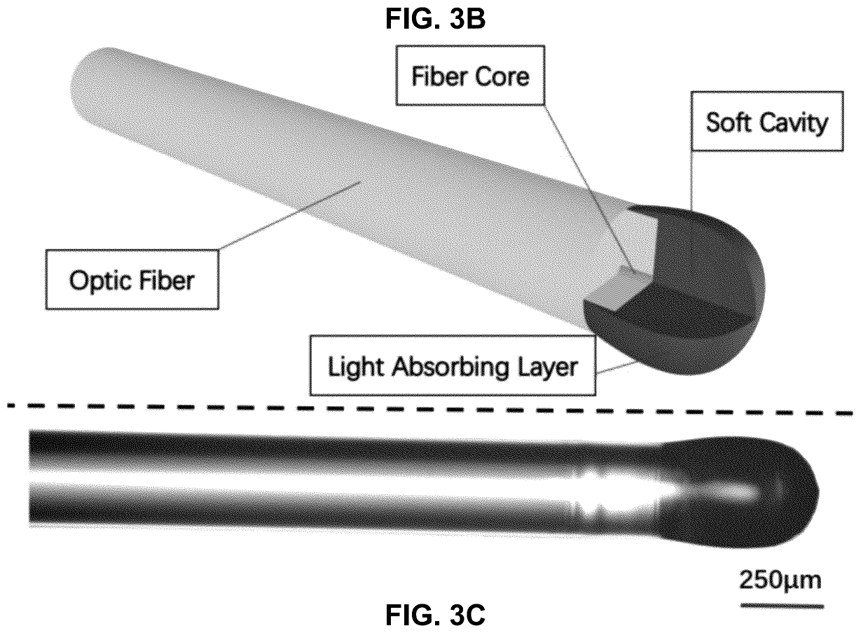

To address these challenges, a fiber-optic sensor is reported that can be deployed through a standard 250-μm injection needle (25 gauge) for minimally invasive measurements of deep-tissue biomechanical properties in vivo (FIGS. 1, 2, and 3). The sensor's ability to provide distinct readouts of muscle stiffness when the hind limb of a rat is relaxed and stretched is demonstrated. To ensure minimal tissue damage and distortion, optical proximity sensing has been integrated within the same fiber for real-time, precise control of the sensor position. To facilitate clinical translation, the sensor has been designed to be disposable and autoclavable and a strategy for mass production has been developed.

More specifically, a fiber-optic sensor that enables highly localized measurements of biomechanical properties in deep tissue has been developed. The sensor incorporates a white-light interferometer to measure the surrounding pressure, based on which tissue stiffness and viscosity can be derived. Moreover, the sensor has an overall diameter of 140 μm and can be deployed through a standard 25-gauge injection needle to gain access to deep tissue with minimal invasiveness. To minimize the risk of over-protrusion and tissue damage, optical coherent detection of backscattered light has been implemented through the same fiber for real-time monitoring of the sensor's relative location with respect to the targeted tissue. To facilitate clinical translation, the sensor has been designed to be disposable and autoclavable and have developed a strategy for mass production has been developed. One immediate and impactful application of the fiber-optic sensor is to assess the biomechanical properties of muscle trigger points, which play an essential role in myofascial pain. This approach holds promise for the development of quantitative biomarkers to improve pain management. Towards this goal, initial experiments have been conducted to validate the technical feasibility by inserting the sensor into a hind limb muscle of a rat through a 25-gauge needle. The results demonstrate the sensor's capability to obtain distinct stiffness readouts when the limb was relaxed and stretched.

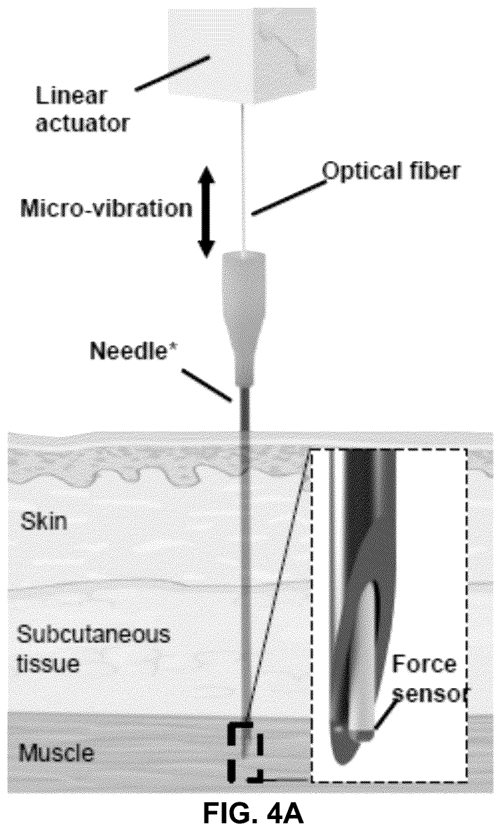

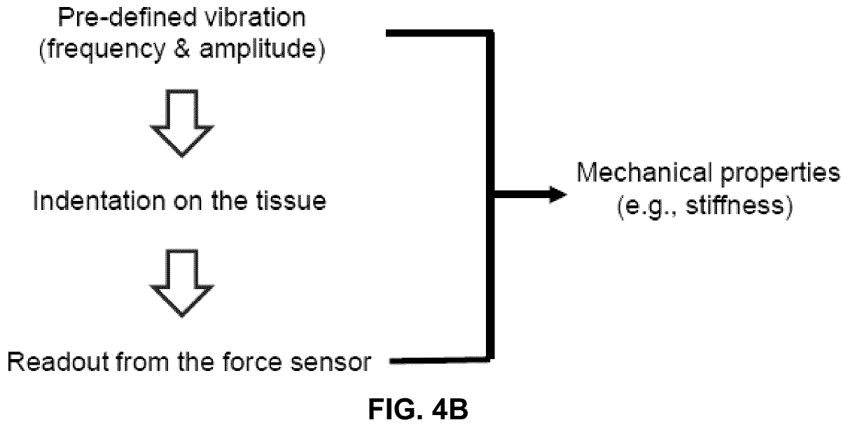

To measure muscle tissue stiffness, a fiber-optic micro-palpation device was used, shown illustrated in FIG. 4A. The device includes a linear actuator, an optical fiber configured to transmit micro-vibrations from the linear actuator to the tissue at the opposite end of the optical fiber, a needle configured to penetrate the skin and deliver the optical fiber to the desired tissue, including, but not limited to, a muscle layer of a subject as illustrated in FIG. 4A, and a force sensor on the distal tip of the optical fiber opposite the linear actuator (FIG. 4A). It is to be noted that a similarly dimensioned needle is commonly used in myofascial pain management (e.g., drug injection, dry needling), making this device no more invasive than the current standard of care. The method uses a pre-defined vibration (frequency and amplitude) delivered from the linear actuator to the tissue via the optical fiber, causing periodic indentations of the tissue, causing periodic fluctuations of a readout from the force sensor positioned on the distal tip of the optical fiber, which is analyzed to estimate mechanical properties of the tissue such as tissue stiffness (FIG. 4B).

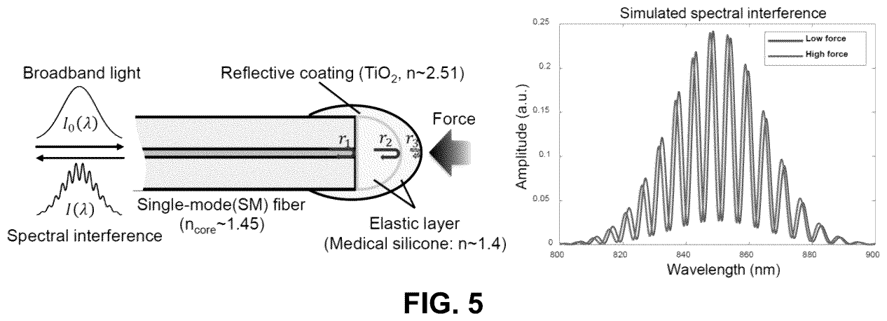

In these experiments, the force sensor positioned at the distal tip of the optical fiber was a Fabry-Perot (FP) force sensor configured to provide spectral interference patterns generated by potions of light delivered by the optical fiber and reflected by an interface between the distal tip of the optic fiber and a TiO₂ reflective layer of the force sensor, and the interface between a distal silicone elastic layer and the reflective TiO₂ coating (FIG. 5, left). The spectral interference can be characterized according to the equation below:

$$I ( λ ) = {I}_{0} ( λ ) [ ( {r}_{1} + {r}_{2} ) - 2 \sqrt{{r}_{1} {r}_{2}} V cos ( \frac{2 π}{λ} \cdot OPD + ϕ ) ]$$

wherein V=fringe visibility, OPD=optical path length difference, and φ=initial phase. As illustrated in FIG. 5, left, the optical path length difference (OPD) is reduced as the tip of the force sensor is compressed, causing alterations in the spectral interference produced by the force sensor, as illustrated in FIG. 5, right.

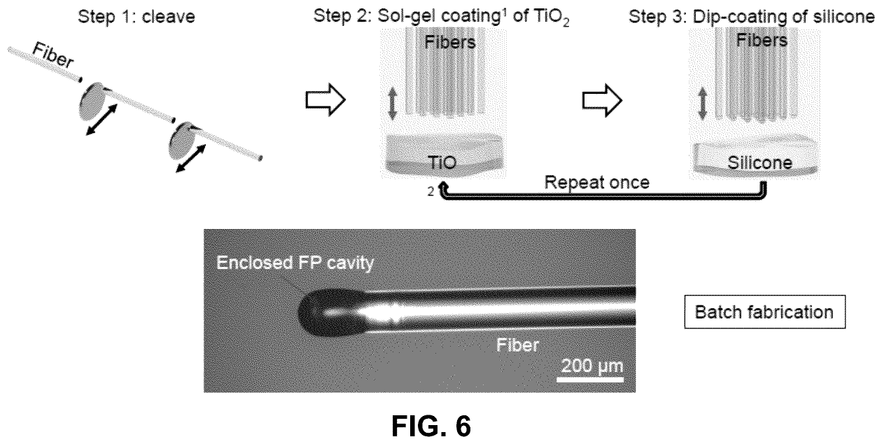

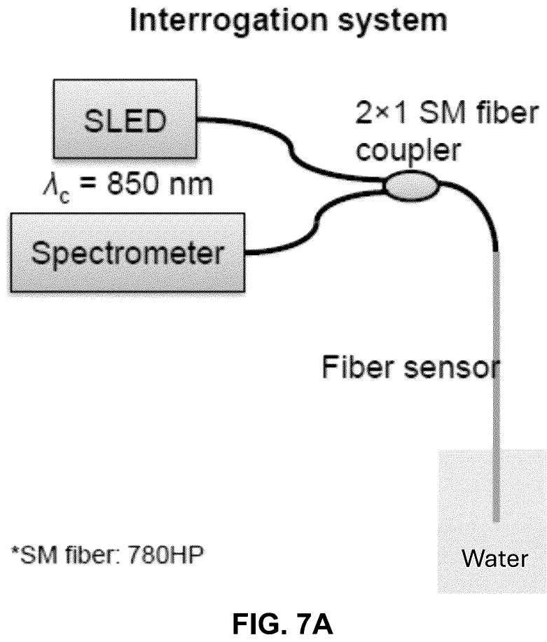

In these experiments, the force sensor is fabricated in batches by cleaving optical fibers, coating the distal fiber tips in TiO₂ using a sol-gel method, and then further tip coating the tips in silicone (FIG. 6).

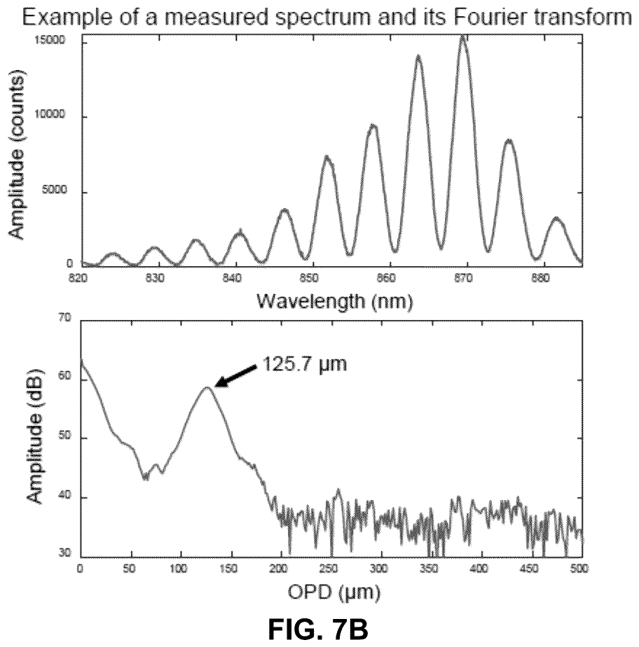

The tissue elasticity measurement system used in these experiments includes fiber sensor described above as well as a superluminescent light-emitting diode (SLED) as a light source and a spectrometer to receive the interference spectra from the fiber sensor described above. The SLED and spectrometer are optically coupled into an optical fiber through a 2×1 fiber coupler, and the fiber sensor is coupled optically coupled to the opposite end of the 2×1 fiber coupler, as shown in FIG. 7A. An illustrative example of a measured interference spectrum and its Fourier transform as obtained using the tissue elasticity measurement system by the from the device is shown in the upper and lower plots of FIG. 7B, respectively.

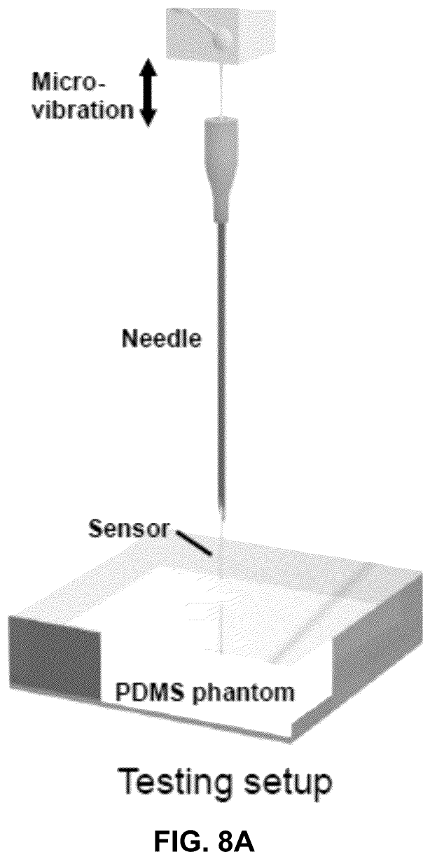

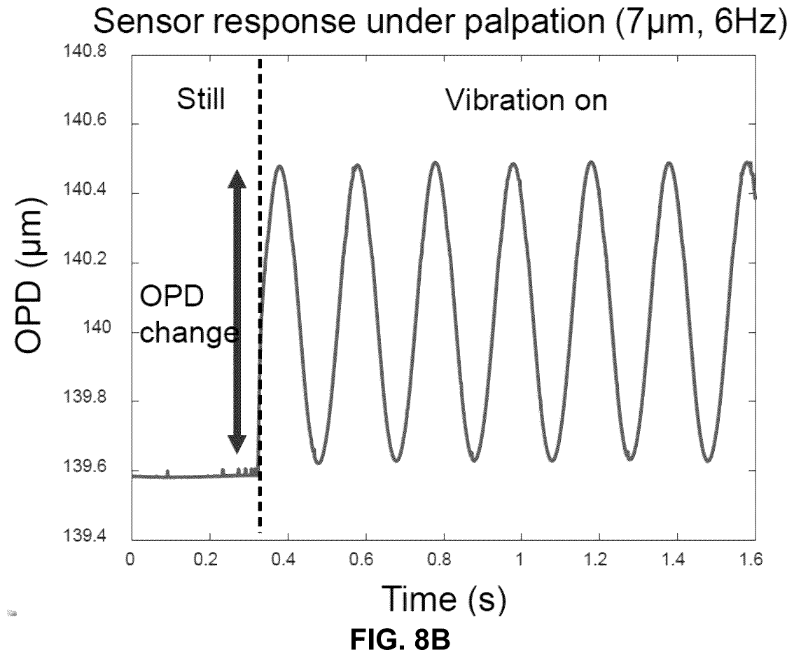

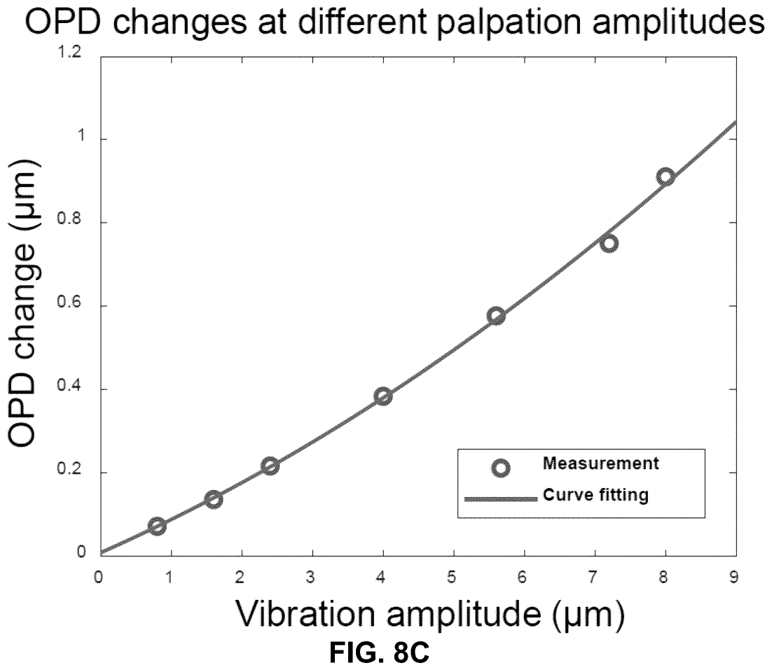

The response of the tissue elasticity measurement system was validated by using measurements obtained using a PDMS phantom test as illustrated in FIG. 8A. The sensor response under a palpation of 7 μm depth at a frequency of 6 Hz is shown in FIG. 8B, and the OPD changes at different palpation amplitudes are shown in FIG. 8C. The device exhibits a linear response at small amplitudes, and there is an anticipated larger linear range in muscle tissue, with stiffness around 101 kPa, compared to 101 MPa of PDMS.

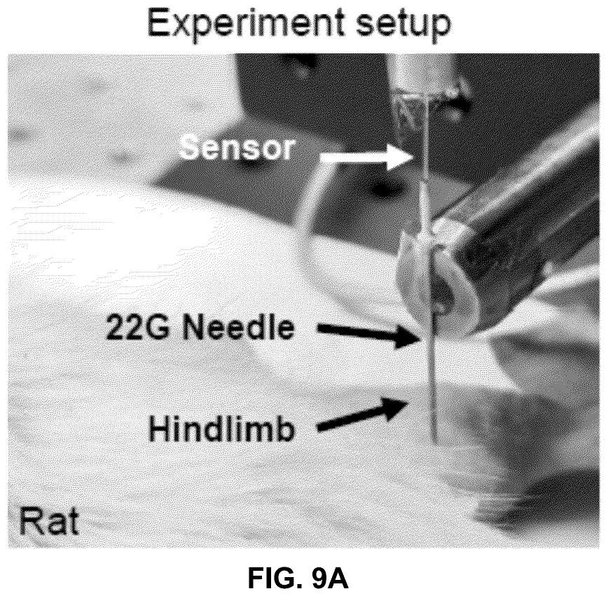

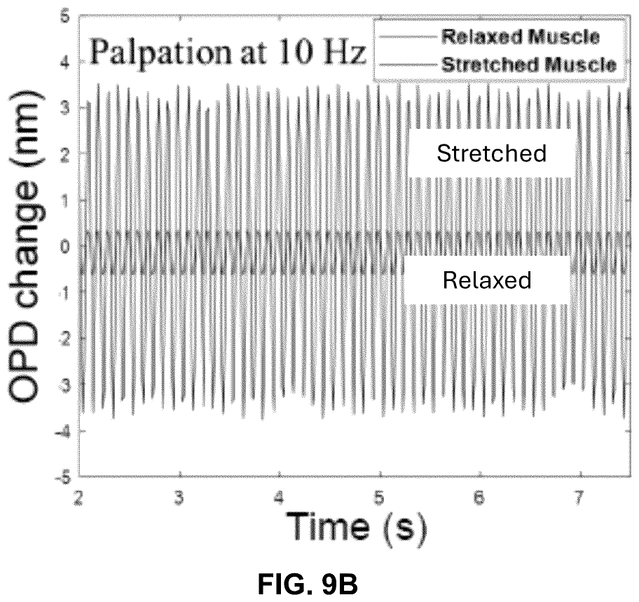

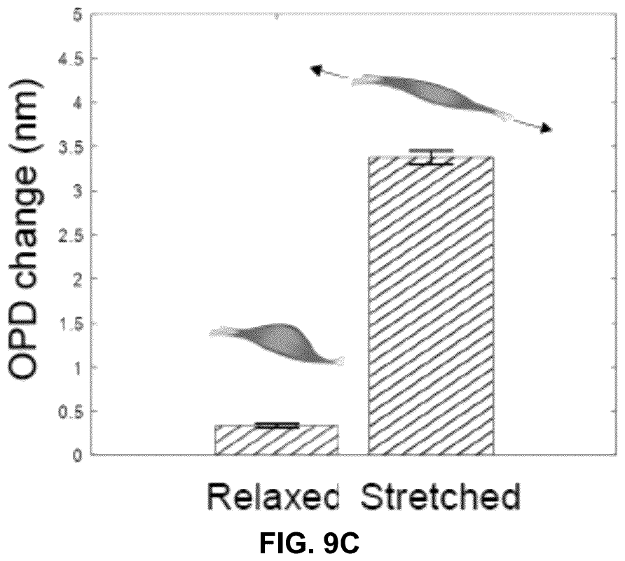

In vivo validation in rats was performed using a similar experimental setup (FIG. 9A). Palpation was performed at 10 Hz in relaxed and stretched muscles (FIG. 9B), and the sensor readouts (e.g., OPD change) at different muscle states were distinguishable (FIG. 9C).

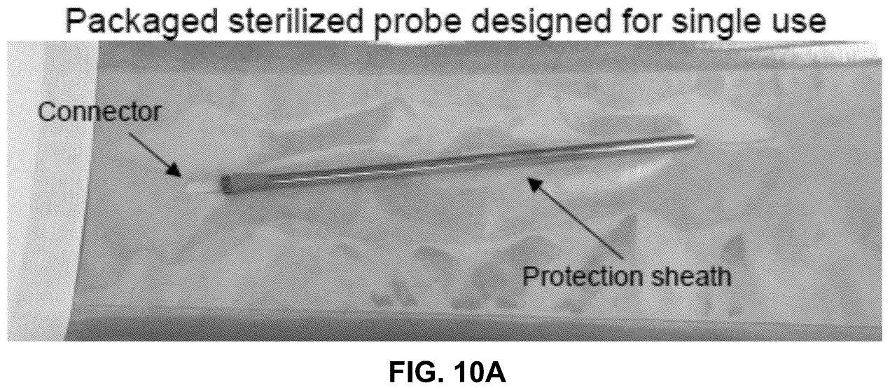

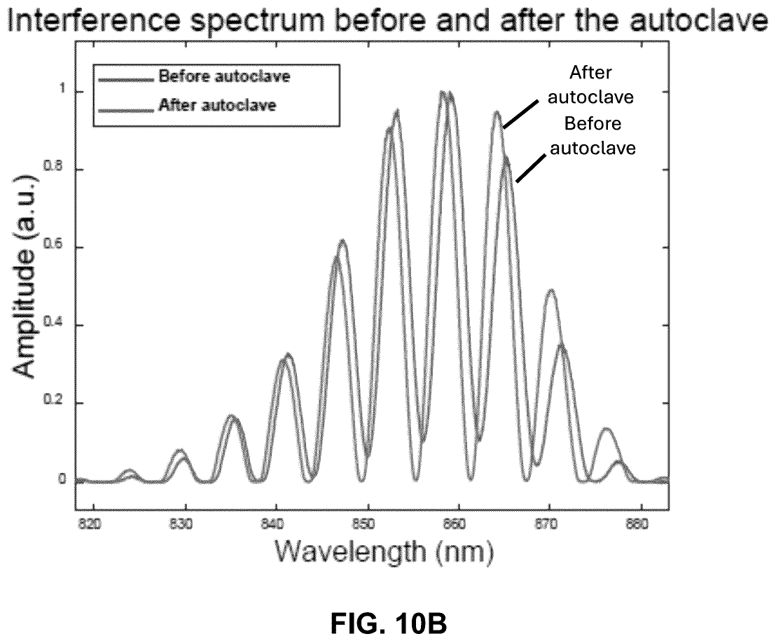

Further, sterilization by autoclave was integrated into the fabrication process for clinical translation. A packaged sterilized probe designed for single use is illustrated in FIG. 10A. Minute changes in the interference pattern after autoclave was observed as illustrated in FIG. 10B, demonstrating that the probe is compatible with standard autoclave sterilization.

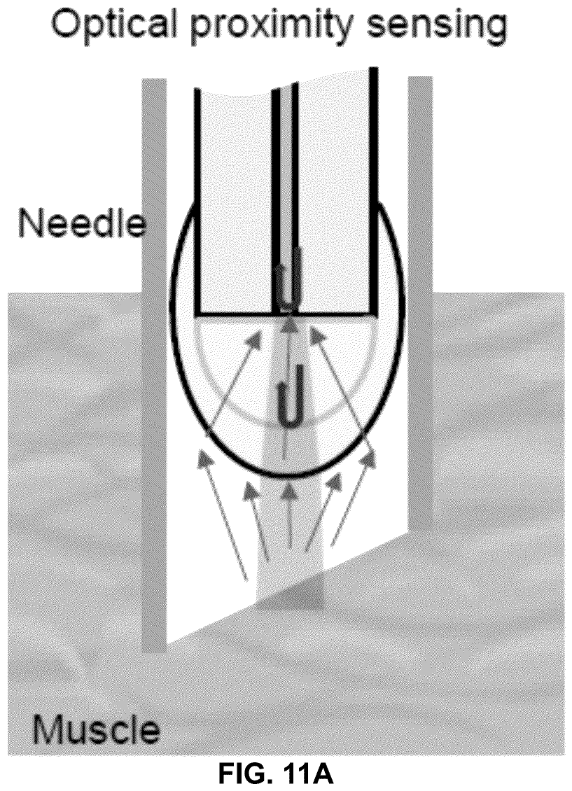

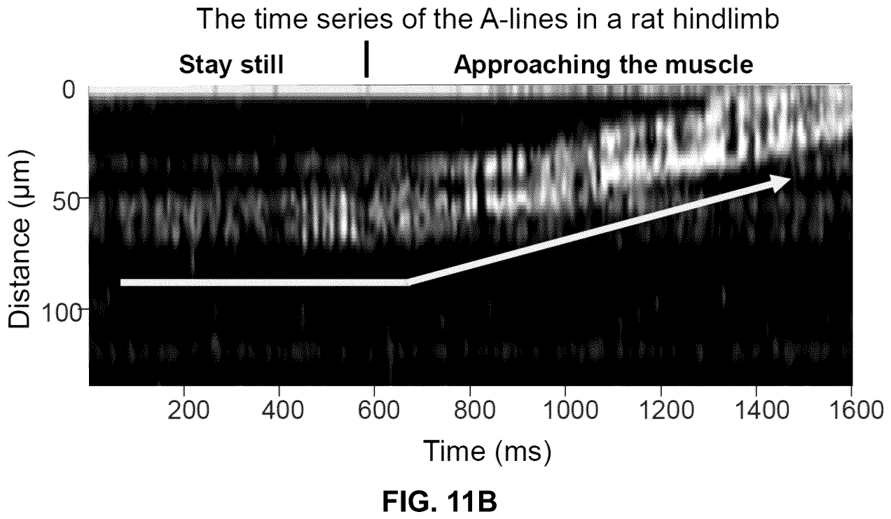

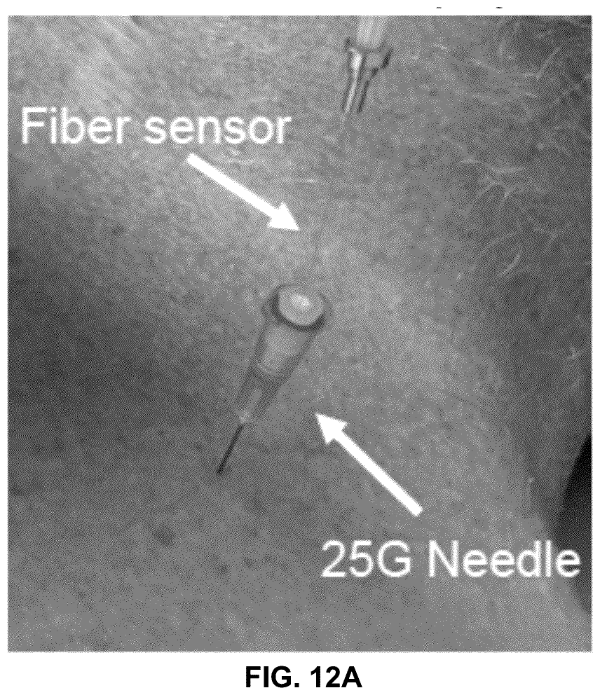

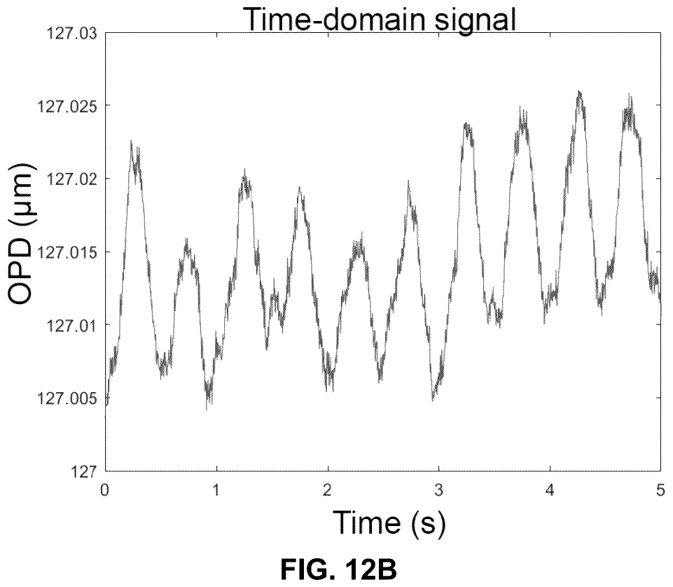

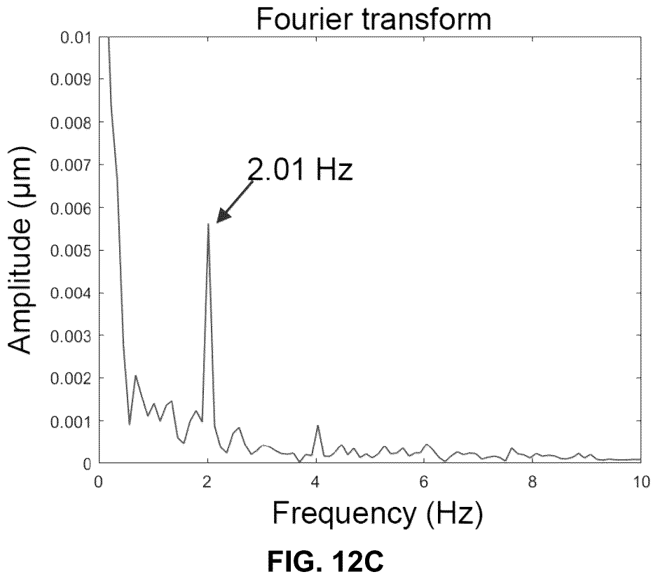

Proximity sensing in a rat hind limb using the same fiber force sensor was explored for further clinical translation applications (FIG. 11A). The time series of the A-lines in a rat hindlimb is summarized in FIG. 11B, demonstrating real-time distance feedback with micro-meter accuracy. Further, the technical feasibility of clinical testing was performed on a human subject (FIG. 12A). A 2 Hz sinusoidal palpation with an amplitude of 2 μm was performed for 5 seconds, and the time signal and its Fourier transform can be presented in FIGS. 12B and 12C, respectively.

The results of these experiments demonstrated the feasibility of using a minimally-invasive fiber-optic approach to measure deep-tissue mechanical properties. This approach was validated in rat muscle by demonstrating distinct readouts between the relaxed and stretched rat muscle states. The sensor was demonstrated to be autoclavable to facilitate the clinical translation. Clinical feasibility of the approach was demonstrated with a healthy human volunteer.

### Example 2—Reduced Diameter Optical Fiber Force Sensor

To demonstrate and validate the feasibility of reducing the diameter of the optical fiber stiffness-sensing probe similar to the probe described in Example 1, the following experiments were conducted.

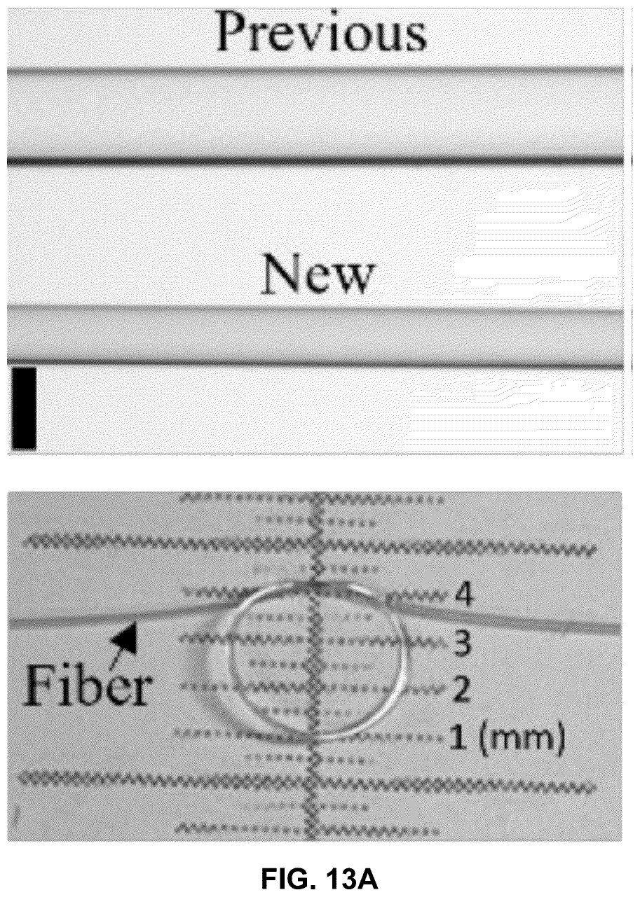

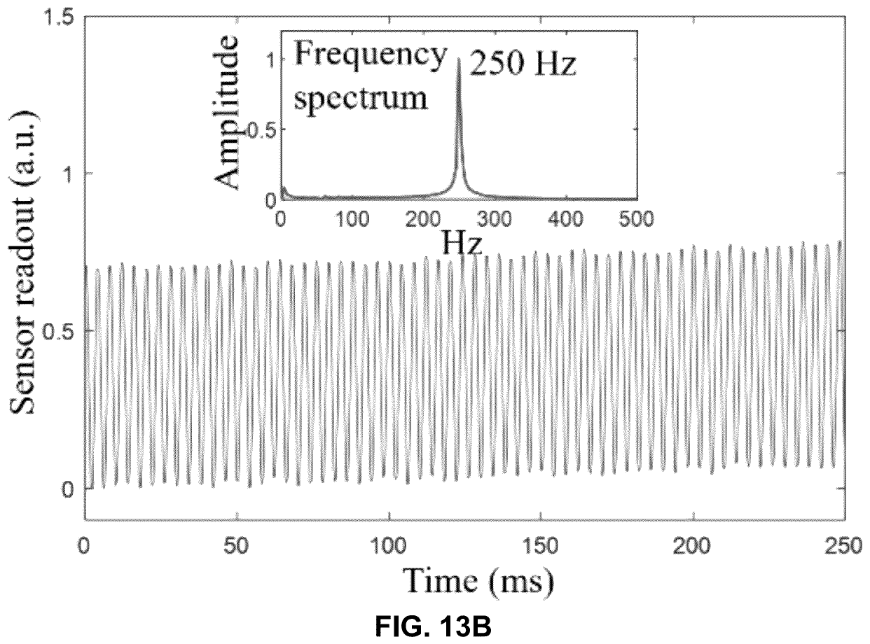

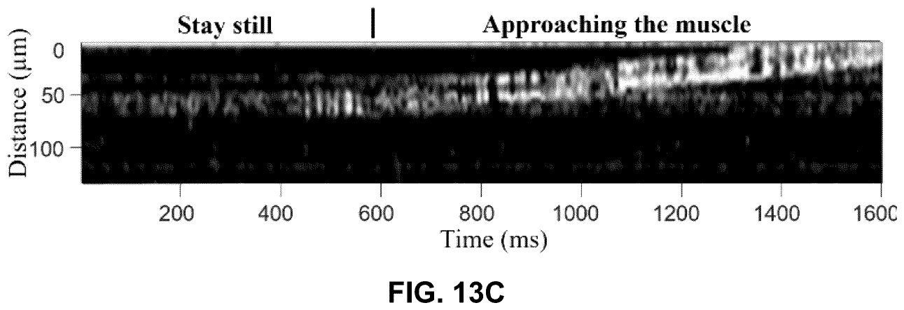

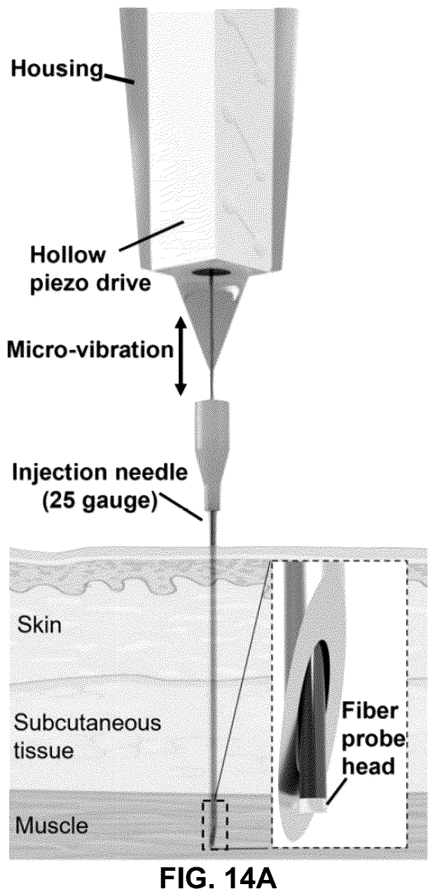

The diameter of the stiffness-sensing probe described in Example 1 was reduced from 250 μm to 150 μm by replacing the silicone outer coating with thin and strong polyimide, facilitating the insertion of the probe into a 25-G injection needle (inner diameter: ˜260 μm) as illustrated in FIG. 13A. A high-speed interrogation system to deliver higher frequency stimulation (>250 Hz compared to 50 Hz in the previous design of Example 1) was also developed (FIG. 13B). The ability to probe higher frequency response promotes a comprehensive characterization of the muscle biomechanics, including the frequency dependence of stiffness and viscosity.

A real-time insertion guide was also developed. The measurements of the optical fiber stiffness sensor rely on physical contact with the muscle tissue and therefore is intrinsically blind during insertion of the probe until the distal tip contacts the muscle. To prevent under or over-insertion of the probe, optical-coherent proximity sensing was integrated into the stiffness probe to provide real-time feedback on the fiber tip's distance to the targeted tissue. The fiber was used to collect the sensing light backscattered from the tissue and quantify its delay via interferometry, where the distance information can be extracted in real time (FIG. 13C). This allows us to precisely control the position of the stiffness-sensing fiber and promote efficiency, repeatability, and safety in the trials.

### Example 3—Multifunctional Fiber Probe

In some aspects, the optical fiber-based force probe described above may be incorporated into a multifunctional system configured to perform multiple tissue imaging and mechanical property measurements using the same optical fiber-based probe.

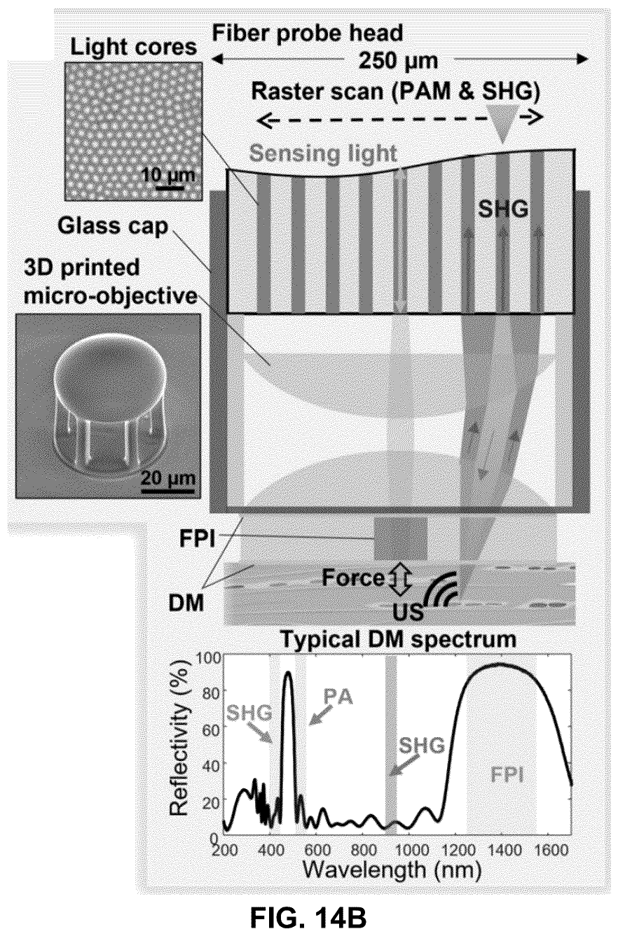

The techniques described in the present disclosure are further expanded by integrating multiple individual functions into a single fiber to meet the requirement on the size compatibility with a chemical injection needle. As shown in FIG. 14A, the probe consists of a housing and a multi-functional fiber that can be inserted into the injection needle. The housing contains a hollow piezo (PK4FXH3P2, Thorlabs), which drives the fiber probe tip back and forth at 100 Hz with a fixed displacement (50 μm) for tissue stiffness sensing. The multi-functional fiber consists of an image guide for light delivery and collection, a micro-objective for light focusing, and an FPI cavity for ultrasound detection and stiffness sensing (FIG. 14B). An image guide (˜220 μm) is selected to ensure the size compatibility with the 25-G injection needle (250 μm). The image guide has 3,000 cores uniformly distributed over the cross-section. Photoacoustic (PAM) and SHG images can be acquired by scanning the light beam core by core and recording the PAM and SHG signals at each location. A micro-objective can be 3D printed on the end surface of the image guide with the in-house two-photon polymerization technique to provide light focusing for both PAM and SHG imaging (FIG. 14B). The emitted SHG signal is coupled back to the image guide through the same micro-objective. For ultrasound detection and stiffness sensing, a thin waveguide layer (˜50 μm) can be 30 printed at the bottom of the micro-objective and sandwiched by two multi-layer dielectric mirrors to form an FPI cavity. By following preliminary tests, the two mirrors will be designed to selectively reflect the interrogation light for the FPI detection while transmitting to the excitation light for PAM and SHG imaging (FIG. 14B). The ultrasound wave in PAM and the external force in stiffness sensing both alter the physical properties of the FPI cavity and thus the reflectivity of the interrogation light. By measuring the changes in the returning power of the interrogation light, both the photoacoustic signals and tissue's elasticity response can be derived because the two signals are readily separable in the frequency domain (107 Hz vs. 102 Hz). A thin glass cap, assembled in-house with a glass capillary (15 μm wall thickness) and a cover glass (40 μm), are used to mechanically isolate the micro-objective to minimize its deformation during the stiffness sensing.

Optical System Associated with the Fiber Probe for Multi-Functional Interrogation

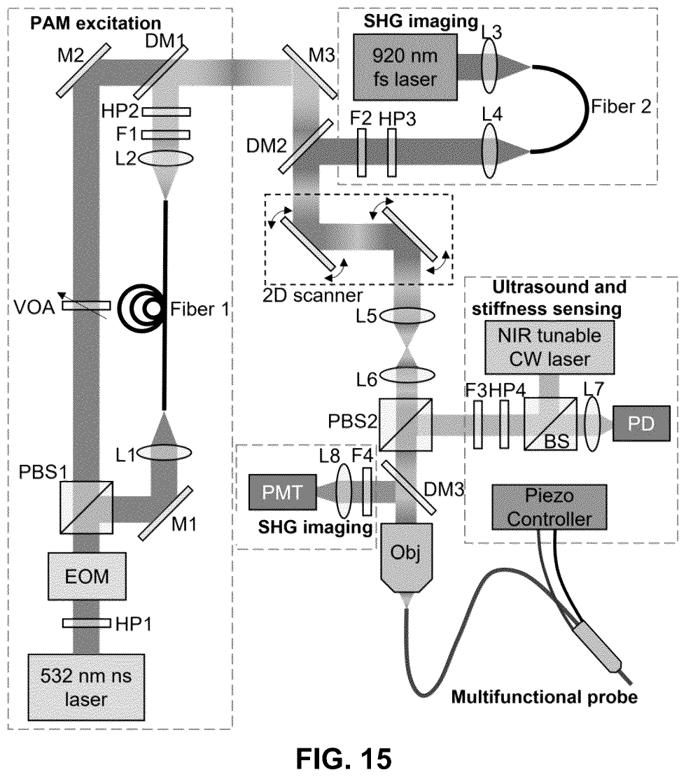

The multifunctional optical fiber probe may be used in a system that provides for three types of sensing modalities, as illustrated in FIG. 15.

Referring again to FIG. 15, the system will provide for photoacoustic microscopy (PAM) by incorporating PAM excitation based on a self-developed, dual-wavelength Raman laser. The collimated beam from a 532-nm ns-pulsed laser will go through a half-wave plate, an electro-optic modulator (EOM), and a polarizing beamsplitter (PBS). The EOM voltage will be altered at every pulse to switch the beam between two light paths, with or without the stimulated Raman scattering (SRS)-based wavelength conversion in a polarization-maintaining (PM) single-mode (SM) optical fiber (Fiber 1). The 532-nm light will be redshifted by the SRS, and the 558-nm Stokes component will be selected from the output with a bandpass filter (F1). Light from the two paths will be merged by a dichroic mirror (DM1), collimated into a 20 galvo scanner, and projected onto the back focal plane of an objective lens (Obj) through a relay lens pair (LS and L6) for raster scan across all cores of the image guide.

Referring again to FIG. 15, the system will provide for 2nd-harmonic generation (SHG) imaging. The SHG signal will be excited with 920-nm ps laser pulses generated by broadening the fs-pulsed laser output with a PM-SM fiber (Fiber 2). Picosecond pulses have been demonstrated for muscle SHG imaging and experience negligible chromatic dispersion in a short image guide (1 m in our design). The use of 920 nm pulses for the SHG signal excitation avoids possible confounding by tissue autofluorescence. The fs SHG excitation and ns PAM excitation will be combined via a dichroic mirror (DM2) and launched into the same scanning setup. The SHG signal will be coupled back to the image guide and detected by a photomultiplier tube (PMT) after spectrally filtered by a dichroic mirror (DM3) and a bandpass filter (F4).

Referring again to FIG. 15, the system will further provide for Fabry-Pérot interferometer (FPI)-based ultrasound detection and stiffness sensing (described above). A near-infrared (NIR) tunable CW laser will be used to interrogate changes in the FPI cavity. Its wavelength will be tuned to the inflection point of the FPI to optimize its sensitivity. The CW laser output will be combined with the PAM and SHG excitation light via a PBS by adjusting their polarization states using half-wave plates (HP1-4). The combined beam will be focused into the selected core through the same objective. Light reflected from the FPI will be recorded by a photodiode (PD) after the residual photoacoustic and fluorescent excitation and the fluorescent emission are removed (DM3 and F3). Signals from the PD and PMT will be recorded by a data-acquisition card. The lasers, EOM, scanner, and piezo controller will be coordinated by a Field Programmable Gate Array (FPGA).

What is claimed is:

1. A fiber-optic biomechanical sensing system, the system comprising:
a. a biomechanical sensor comprising:
i. an optical fiber comprising opposed proximal and distal ends; and
ii. a deformable Fabry-Perot interferometer optically coupled to the distal end of the optic fiber;
b. a light source optically coupled to the biomechanical sensor at the proximal end of the optic fiber;
c. a vibrational indentation module mechanically coupled to the biomechanical sensor, the vibrational indentation module configured to periodically advance and retract the biomechanical sensor along a proximal-distal axis at a predetermined frequency and displacement; and
d. a spectrometer optically coupled to the fiber optic of the biomechanical sensor, the spectrometer configured to receive a plurality of optical signals from the deformable Fabry-Perot interferometer;
wherein the optical signals encode interference patterns generated by the deformable Fabry-Perot interferometer in response to the periodic displacements of the biomechanical sensor by the vibrational indentation module.

2. The system of claim 1, wherein the optical fiber is a polymer-coated glass optical fiber.

3. The system of claim 1, wherein the Fabry-Perot interferometer comprises:
a. a deformable cavity comprising a proximal surface optically coupled to the distal end of the fiber optic and a distal surface opposite the proximal surface;
b. a first reflective coating covering at least a portion of the proximal surface, the first reflective coating configured to reflect a first portion of light from the light source at the distal end of the optic fiber and further configured to transmit a second portion of light from the light source to the distal surface of the deformable cavity; and
c. a second reflective coating covering at least a portion of the distal surface of the deformable cavity, the second reflective coating configured to reflect the second portion of light back through the first reflective coating and optic fiber;
wherein the reflected first and second portions of light form an interference pattern propagating through the optic fiber to the spectrometer.

4. The system of claim 1, wherein the deformable cavity comprises a soft and transparent material selected from polydimethylsiloxane (PDMS), a hydrogel, and an optical adhesive.

5. The system of claim 1, wherein the first and second reflective coatings comprise at least one dielectric material.

6. The system of claim 5, wherein the dielectric materials of the first and second reflective coatings are selected independently from titanium dioxide (TiO₂), zinc oxide (ZnO), and any combination thereof.

7. The system of claim 5, wherein the first and second reflective coatings comprise thin dielectric coatings formed using a sol-gel method to increase light reflectivity.

8. The system of claim 1, wherein the light source comprises a broadband light source selected from a light-emitting diode (LED), a superluminescent diode (SLED), and a lamp.

9. The system of claim 1, wherein the interference patterns generated by the deformable Fabry-Perot interferometer are modulated by changes in a cavity thickness between the proximal and distal surfaces of the deformable cavity induced by compressions associated with the periodic displacements of the biomechanical sensor by the vibrational indentation module.

10. The system of claim 1, wherein the biomechanical sensor is disposable and autoclavable for sterilization to facilitate clinical translation.

11. The system of claim 1, wherein the biomechanical sensor is mass-producible.

12. The system of claim 1, wherein the vibrational indentation module comprises a piezo actuator operatively coupled to a controller, the controller comprising a voltage modulator.

13. The system of claim 12, wherein the voltage modulator is configured to produce a modulated voltage at a predetermined frequency or shape to actuate the periodic displacements of the piezo actuator.

14. The system of claim 1, wherein the spectrometer is a high-speed spectrometer.

15. The system of claim 1, wherein the biomechanical sensor is further configured to be deployed through a standard 25-gauge injection needle.

16. The system of claim 1, wherein the biomechanical sensor further comprises a cross-sectional diameter of about 140 μm.

17. A method to measure at least one biomechanical property of a tissue, the method comprising:
a. providing a fiber-optic biomechanical sensing system, the system comprising:
i. a biomechanical sensor comprising:
1. a fiber optic comprising opposed proximal and distal ends; and
2. a deformable Fabry-Perot interferometer optically coupled to the distal end of the optic fiber;
ii. a light source optically coupled to the biomechanical sensor at the proximal end of the optic fiber;
iii. a vibrational indentation module mechanically coupled to the biomechanical sensor, the vibrational indentation module configured to periodically advance and retract the biomechanical sensor along a proximal-distal axis at a predetermined frequency and displacement; and
iv. a spectrometer optically coupled to the fiber optic of the biomechanical sensor, the spectrometer configured to receive a plurality of optical signals from the deformable Fabry-Perot interferometer, wherein the optical signals encode interference patterns generated by the deformable Fabry-Perot interferometer in response to the periodic displacements of the biomechanical sensor by the vibrational indentation module;
b. positioning the deformable Fabry-Perot interferometer of the biomechanical sensor against the tissue;
c. periodically advancing and retracting the biomechanical sensor along a proximal-distal axis at a predetermined frequency and displacement while delivering light from the light source to generate a series of interference patterns;
d. detecting the series of interference patterns at the spectrometer; and
e. transforming the series of interference patterns into the at least one biomechanical property of the tissue.

18. The method of claim 17, wherein the at least one biomechanical property is selected from stiffness, viscosity, and any combination thereof.

19. The method of claim 17, wherein the at least one biomechanical property is recorded in real-time.

20. The method of any one of claim 17, wherein positioning the deformable Fabry-Perot interferometer of the biomechanical sensor against the tissue comprises deploying the biomechanical sensor through a standard injection needle.
